(19) United States
(12) Patent Application Publication
(10) Pub. No.: US 2020/0384436 A1
(43) Pub. Date: Dec. 10, 2020

(54) METHODS FOR FABRICATING HIGH RESOLUTION DNA ARRAY AND ITS APPLICATION IN SEQUENCING

(71) Applicant: CENTRILLION TECHNOLOGIES, INC., Palo Alto, CA (US)

(72) Inventors: Filip CRNOGORAC, Redwood City, CA (US); Glenn MCGALL, Palo Alto, CA (US); Paul DENTINGER, Sunol, CA (US); T. Scott POLLOM, Menlo Park, CA (US); Wei ZHOU, Saratoga, CA (US)

(21) Appl. No.: 16/959,578

(22) PCT Filed: Jan. 5, 2019
(86) PCT No.: PCT/US2019/012444

§ 371 Date: Jul. 1, 2020

Related U.S. Application Data
(60) Provisional application No. 62/614,307, filed on Jan. 5, 2018.

(51) Int. Cl. B01J 19/00; C07H 21/04; C40B 50/18

(52) U.S. Cl. CPC B01J 19/0046; C07H 21/04; C40B 50/18; B01J 2219/00432; B01J 2219/00722; B01J 2219/00596; B01J 2219/00659; B01J 2219/00711; B01J 2219/00529

(57) ABSTRACT

The present disclosure provides methods and processes for forming a pattern of oligonucleotides on a microarray. A method for forming a pattern of oligonucleotides on a microarray may include forming a photoresist layer by applying a photoresist composition onto an underlying layer of a substrate, exposing a dose of light through a patterned mask onto the substrate, and removing protective groups on a section of the plurality of functional groups within at least one exposed region of the substrate, wherein the photoresist composition comprises a photoacid generator, an acid scavenger and a photosensitizer, wherein the underlying layer comprises a plurality of functional groups protected by protective groups; thereby forming a pattern on the substrate, wherein the pattern comprises the at least one exposed region, and wherein the at least one exposed region is no more than 1 micrometer in at least one dimension.

### CROSS REFERENCE

This application claims priority to U.S. Provisional Patent Application No. 62/614,307, filed Jan. 5, 2018, which is herein incorporated by reference in its entirety for all purposes.

### STATEMENT REGARDING FEDERALLY SPONSORED RESEARCH OR DEVELOPMENT

This invention was made with support of the United States government under grant numbers 1R43HG008582-01 and 5R43HG008582-02 awarded by the National Institutes of Health. The government has certain rights in the invention.

### BACKGROUND

Significant advances in biological sciences have led to unprecedented advances in understanding the mechanisms of life, health, disease and treatment. In particular, genomic sequencing is used to obtain biomedical information in areas including diagnostics, prognostics, biotechnology, personalized medicine, and forensics. High-density nucleic acid microarrays have seen extensive use in a range of applications for genomic sequence analysis, including the detection and analysis of mutations and polymorphisms, cytogenetics (copy number), nuclear proteomics, gene expression profiling, and transcriptome analysis.

Biomolecule arrays with biomolecules immobilized on solid support have been employed in the fields of molecular biology. Biomolecules immobilization may provide advantages, such as, allowing for multiplexing of samples and location addressable identification of signals for target molecules. Creating biomolecule arrays, including oligonucleotide arrays, on a flat solid support, have attracted a lot of research.

In particular, microarrays (DNA chips) are important tools for high-throughput analysis of biomolecules. One key component for microarray fabrication is the chemistry employed to immobilize DNA probes. Other factors to be considered involve the hydrophilicity of the surface, the accessibility of the surface-bound probes, the density of the probes, and the reproducibility of the underlying chemistry processes. A. Sassolas et al., Chem. Rev. (2008) 108(1):109-39. One method to construct oligonucleotide microarrays is the in situ syntheses of oligonucleotides on the chip surface using either photolithographic methods or deposition methods. D. Sethi et al. Bioconjugate Chem. (2008) 19(11):2136-43.

### SUMMARY

While recent advancement in nucleic acid sequencing technologies has greatly improved the routine detection of nucleic acids, including, for example, a deoxyribonucleic acid (DNA) or a ribonucleic acid (RNA), resolving the precise sequences of large biomolecules is still a major challenge. Nucleic acid sequencing is a fundamental technology essential to modern technologies, including, for example, personalized medicine. Despite rapid advances in DNA sequencing technologies in recent years, there are still needs for improved DNA and RNA sequencing methods, including sequencing long nucleic acids.

Provided herein are methods, systems and compositions for the preparation of oligonucleotide microarrays with features no more than 1 μm in at least one dimension.

In an aspect, the present disclosure provides a method for forming a pattern of oligonucleotides on a microarray, comprising: (a) forming a photoresist layer by applying a photoresist composition onto an underlying layer of a substrate, wherein the photoresist composition comprises a photoacid generator and a photosensitizer, wherein the underlying layer comprises a plurality of functional groups protected by protective groups; (b) exposing a dose of light through a patterned mask onto the substrate; and (c) removing protective groups on a section of the plurality of functional groups within at least one exposed region of the substrate; thereby forming a pattern on the substrate, wherein the pattern comprises the at least one exposed region, and wherein the at least one exposed region is no more than 1 micrometer in at least one dimension.

In some embodiments of aspects provided herein, the functional groups are amino or hydroxyl groups. In some embodiments of aspects provided herein, the method further comprises: (d) contacting the functional groups within the at least one exposed region of the substrate with a first nucleotide reagent, thereby coupling a fraction of the functional groups within the at least one exposed region of the substrate with a first nucleotide. In some embodiments of aspects provided herein, the method further comprises (e) exposing another dose of light through another patterned mask onto the substrate; (f) removing protective groups on another section of the plurality of functional groups within at least another exposed region of the substrate; thereby forming another pattern on the substrate, wherein the another pattern comprises the at least another exposed region, and wherein the at least another exposed region is no more than 1 micrometer in at least one dimension. In some embodiments of aspects provided herein, the method further comprises: (g) contacting the functional groups within the at least another exposed region of the substrate with a second nucleotide reagent, thereby coupling another fraction of the functional groups within the at least another exposed region of the substrate with a second nucleotide. In some embodiments of aspects provided herein, the first nucleotide is different from the second nucleotide. In some embodiments of aspects provided herein, the at least one exposed region is different from the at least another exposed region.

In some embodiments of aspects provided herein, the method further comprises: (e) forming another photoresist layer by applying another photoresist composition onto the substrate, wherein the another photoresist composition comprises another photoacid generator and another photosensitizer, wherein the underlying layer comprises a plurality of functional groups protected by protective groups; (f) exposing another dose of light through another patterned mask onto the substrate; (g) removing protective groups on another section of the plurality of functional groups and/or a nucleotide protective group on a nucleotide functional group on the first nucleotide within at least another exposed region of the substrate; thereby forming another pattern on the substrate, wherein the another pattern comprises the at least another exposed region. In some embodiments of aspects provided herein, at least another exposed region is no more than 1 micrometer in at least one dimension. In some embodiments of aspects provided herein, the method further comprises (h) contacting the functional groups and/or the nucleotide functional group on the first nucleotide within the at least another exposed region of the substrate with a second nucleotide reagent, thereby coupling another fraction of the functional groups and/or the nucleotide functional group within the at least another exposed region of the substrate with a second nucleotide. In some embodiments of aspects provided herein, the first nucleotide is different from the second nucleotide. In some embodiments of aspects provided herein, the at least one exposed region is different from the at least another exposed region.

In some embodiments of aspects provided herein at least one exposed region is no more than 950 nm, 900 nm, 850 nm, 800 nm, 750 nm, or 700 nm in the at least one dimension. In some embodiments of aspects provided herein, weight percentage of the photosensitizer is substantially the same as weight percentage of the photoacid generator. In some embodiments of aspects provided herein, the weight percentage of the photosensitizer is the same as the weight percentage of the photoacid generator.

In some embodiments of aspects provided herein, the photoresist composition further comprises, an acid scavenger, a matrix and a solvent. In some embodiments of aspects provided herein, the photoresist composition comprises: the photoacid generator: about 2-5% by weight; the photosensitizer: about 2-5% by weight; an acid scavenger: about 0.1-0.5% by weight; a matrix: about 2.5-4.5% by weight; and a solvent: about 85-93.4% by weight. In some embodiments of aspects provided herein, the photoresist composition comprises: the photoacid generator: about 2.5-4.5% by weight; the photosensitizer: about 2.5-4.5% by weight; the acid scavenger: about 0.15-0.35% by weight; the matrix: about 3.0-4.0% by weight; and the solvent: about 86.7-91.8% by weight. In some embodiments of aspects provided herein, weight percentage of the photosensitizer is substantially the same as weight percentage of the photoacid generator. In some embodiments of aspects provided herein, the weight percentage of the photosensitizer is the same as the weight percentage of the photoacid generator.

In some embodiments of aspects provided herein, the pattern and/or the another pattern comprises features of oligonucleotides; and wherein the smallest size of the features of oligonucleotides is no more than 1 μm in at least one dimension. In some embodiments of aspects provided herein, smallest size of the features of oligonucleotides is no more than 950 nm, 900 nm, 850 nm, 800 nm, 750 nm, or 700 nm in the at least one dimension. In some embodiments of aspects provided herein, the features of oligonucleotides is no more than 950 nm, 900 nm, 850 nm, 800 nm, 750 nm, or 700 nm in two dimensions. In some embodiments of aspects provided herein, the method sizes of features of the pattern, features of the another pattern, the at least one exposed region, the at least another exposed region, and/or the features of oligonucleotides are measured by using a super resolution microscopy.

In another aspect, the present disclosure provides a method for forming a pattern of oligonucleotides on a microarray, comprising: (a) activating a photoacid generator in the presence of a photosensitizer in selected regions, thereby producing an acid from the photoacid generator, wherein the substrate comprises a functional group protected by a protective group, wherein the protective group is removed by the acid; (b) contacting the substrate with a reagent for oligonucleotide synthesis; and (c) repeating steps (a) and (b) with another reagent for oligonucleotide synthesis; thereby forming a pattern of oligonucleotides, wherein at least one feature of the pattern of oligonucleotides is no more than 1 μM in at least one dimension.

In some embodiments of aspects provided herein, the method further comprises heating the substrate. In some embodiments of aspects provided herein, the method further comprises directing a light to the selected regions in step (a). In some embodiments of aspects provided herein, a print dose of the light is directed to the selected region. In some embodiments of aspects provided herein, the print dose of the light produce the acid from the photoacid generator. In some embodiments of aspects provided herein, when no more than one-third of the print dose is directed to the selected region, another photoacid generator within the selected region does not produce another acid from the another photoacid generator.

In some embodiments of aspects provided herein, the method further comprises including an acid scavenger in step (a). In some embodiments of aspects provided herein, the method further comprises, prior to step (a), coating the substrate with a photoresist formulation comprising the photoacid generator and the photosensitizer. In some embodiments of aspects provided herein, the photoresist formulation further comprises a matrix and a solvent. In some embodiments of aspects provided herein, the at least one feature of the pattern of oligonucleotides comprises a plurality of features of oligonucleotides.

In some embodiments of aspects provided herein, the selected region, and/or the plurality of features of oligonucleotides are no more than 1 μM in at least one dimension. In some embodiments of aspects provided herein, the selected region, the at least one feature of the pattern of oligonucleotides, and/or the plurality of features of oligonucleotides are no more than 950 nm, 900 nm, 850 nm, 800 nm, 750 nm, or 700 nm in the at least one dimension. In some embodiments of aspects provided herein, the selected region, the at least one feature of the pattern of oligonucleotides, and/or the plurality of features of oligonucleotides are no more than 950 nm, 900 nm, 850 nm, 800 nm, 750 nm, or 700 nm in two dimensions.

In some embodiments of aspects provided herein, step (a) is conducted using a spin coater. In some embodiments of aspects provided herein, step (b) is conducted by using an oligonucleotide synthesizer. In some embodiments of aspects provided herein, weight percentage of the photosensitizer is substantially the same as weight percentage of the photoacid generator. In some embodiments of aspects provided herein, the weight percentage of the photosensitizer is the same as the weight percentage of the photoacid generator.

In still another aspect, the present disclosure provides a photoresist composition comprises: a photoacid generator: about 2-5% by weight; a photosensitizer: about 2-5% by weight; an acid scavenger: about 0.1-0.5% by weight; a matrix: about 2.5-4.5% by weight; and a solvent: about 85-93.4% by weight.

In some embodiments of aspects provide herein, the photoresist composition comprises: the photoacid generator: about 2.5-4.5% by weight; the photosensitizer: about 2.5-4.5% by weight; the acid scavenger: about 0.15-0.35% by weight; the matrix: about 3.0-4.0% by weight; and the solvent: about 86.7-91.8% by weight. In some embodiments of aspects provided herein, weight percentage of the photosensitizer is substantially the same as weight percentage of the photoacid generator. In some embodiments of aspects provided herein, the weight percentage of the photosensitizer is the same as the weight percentage of the photoacid generator.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG” and “FIGs” herein), of which:

FIG. 1 is schematically illustrates an example of oligo zipcode.

FIG. 2 depicts an example of megabase sequencing.

FIG. 3 shows an example of calculated areal image from an ASML PA/60 i-line stepper.

FIG. 4 is illustrates an example of a “contrast” curve measurement.

FIG. 5A shows 1.2 μm lines and spaces (L/S) patterns (2.4 μm pitch) of a formulation for a single base addition using ASML PA/60.

FIG. 5B shows 0.6 μm L/S patterns of a formulation for a single base addition using ASML PA/60.

FIG. 5C shows 0.4 μm L/S patterns of a formulation for a single base addition using ASML PA/60.

FIG. 6 depicts a stochastic optical reconstruction microscopy (STORM) image of 600 nm L/S pattern of a formulation for a single base addition.

FIG. 7 shows dose-response curves of formulation to generate acid.

FIG. 8 illustrates contact lithography dot resolution pattern when using a formulation for base addition.

FIG. 9A depicts a fluorescence image of 700 nm L/S patterns of labeled oligonucleotides printed. FIG. 9B illustrates intensity of fluorescence along a cross section of the patterns shown in FIG. 9B.

FIG. 10 shows a STORM super resolution image of labeled oligonucleotides printed.

### DETAILED DESCRIPTION

While various embodiments of the invention have been shown and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

The human genome has complex structures. These structures may still be difficult to analyze even with the help of DNA sequencing technologies that can read short stretches of DNA fragments. One approach for sequencing very long DNA fragments may be to align long DNA molecules on a zipcode DNA or RNA arrays. The zipcode arrays may comprise spatially defined oligonucleotides or other polymers that can encode positional information, such as, for example, the positional information of the oligonucleotides in reference to the array. These spatially defined oligonucleotides may also be called position-encoded zipcode molecules, zipcode molecules, zipcode DNA or zipcode RNA. These position-encoded zipcode molecules can then react with the aligned long DNA molecules on the array surface to either copy the DNA sequence onto the position-encoded zipcode molecules or attach the zipcode molecules to the neighboring molecules, be it a neighboring zipcode molecule, the aligned long DNA molecule or a fragment of the aligned long DNA molecule. There may be biochemical methods to link these zipcode molecules to localized or aligned DNA sequences on the surface of the array so that when the linked molecules are sequenced, a fragment representing a part of the long DNA molecule is associated with one or more zipcode molecules from the surface of the array. Since the position of the zipcode molecules are known based on the sequences of the zipcode molecules decoded, the positional relationship of DNA fragment sequences within the long DNA molecule can be determined.

As used herein, the term “zipcode” generally refers to a known, determinable, and/or decodable sequence, such as, for example, a nucleic acid sequence (DNA sequence or RNA sequence), a protein sequence, and a polymer sequence (including synthetic polymers, carbohydrates, lipids, etc.), that allows the identification of a specific location of the sequence, e.g., the nucleic acid, in one, two or multiple dimensional spaces. A zipcode can encode the decodable sequence's own location. For example, each of the zipcode may be a nucleic acid (may be many copies in a spatially defined location such as a square feature of any size from about 10 nm to about 1 cm, including for example, no larger than 0.1 μm, no larger than 0.2 μm, no larger than 0.3 μm, no larger than 0.4 μm, no larger than 0.5 μm, no larger than 0.6 μm, no larger than 0.7 μm, no larger than 0.8 μm, no larger than 0.9 μm, no larger than 1 μm, no larger than 1.2 μm, no larger than 1.4 μm, no larger than 1.6 μm, no larger than 1.8 μm, o larger than 2 μm, no larger than 5 μm, no larger than 10 μm, no larger than 20 μm, no larger than 30 μm, no larger than 40 μm, no larger than 50 μm, no larger than 100 μm, no larger than 200 μm, no larger than 500 μm, no larger than 1 mm, no larger than 2 mm, and no larger than 5 mm. Zipcode arrays can be used to detect the distribution of ribonucleic acid (RNA), protein, deoxyribonucleic acid (DNA) or other molecules distribution in two or three dimensional space. These biomolecules can be detected in tissue, cell, organism or non-living systems. If a nucleic acid sequence is a zipcode, the complementary sequence of the nucleic acid sequence can also be a zipcode. In this disclosure, a zipcode and its complementary copy can encode the same position/location on the zipcode array.

The zipcodes can be designed for precision sequence performance, e.g., GC content between 40% and 60%, no homo-polymer runs longer than two, no self-complementary stretches longer than 3, and be comprised of sequences not present in a human genome reference. Zipcodes can be of sufficient length and comprise sequences that can be sufficiently different to allow the identification of each nucleic acid (e.g., oligonucleic acids) or peptides based on zipcode(s) with which each nucleic acid or peptides is associated.

As used in this specification and the appended claims, the singular forms “a,” “an” and “the” include plural referents unless the context clearly dictates otherwise. Thus, for example, reference to “a molecule” includes a plurality of such molecules, and the like.

As used herein, the term “about” or “nearly” generally refers to within +/−15%, 10%, 9%, 8%, 7%, 6%, 5%, 4%, 3%, 2%, or 1% of the designated amount.

As used herein, open terms, for example, “comprise”, “contain”, “include”, “including”, “have”, “having” and the like refer to comprising unless otherwise indicates.

As used herein, the term “embedding” and “a string of synthetic steps” generally refer to a series of active and inactive steps designed for forming an individual polymer on the substrate and can be used interchangeably. For example, in cases where light-directed synthetic methods are employed, the “embedding” refer to a series exposure and non-exposure steps.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

As used herein, the term “substrate” generally refers to a substance, structure, surface, material, means, or composition, which comprises a nonbiological, synthetic, nonliving, planar, spherical or flat surface. The substrate may include, for example and without limitation, semiconductors, synthetic metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes; silicon, silicates, glass, metals and ceramics; wood, paper, cardboard, cotton, wool, cloth, woven and nonwoven fibers, materials and fabrics; nanostructures and microstructures. The substrate may comprises an immobilization matrix such as but not limited to, insolubilized substance, solid phase, surface, layer, coating, woven or nonwoven fiber, matrix, crystal, membrane, insoluble polymer, plastic, glass, biological or biocompatible or bioerodible or biodegradable polymer or matrix, microparticle or nanoparticle. Other example may include, for example and without limitation, monolayers, bilayers, commercial membranes, resins, matrices, fibers, separation media, chromatography supports, polymers, plastics, glass, mica, gold, beads, microspheres, nanospheres, silicon, gallium arsenide, organic and inorganic metals, semiconductors, insulators, microstructures and nanostructures. Microstructures and nanostructures may include, without limitation, microminiaturized, nanometer-scale and supramolecular probes, tips, bars, pegs, plugs, rods, sleeves, wires, filaments, and tubes.

As used herein, the term “nucleic acid” generally refers to a polymer comprising one or more nucleic acid subunits or nucleotides. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double-stranded.

As used herein, the term “adjacent” or “adjacent to,” includes “next to,” “adjoining,” and “abutting.” In one example, a first location is adjacent to a second location when the first location is in direct contact and shares a common border with the second location and there is no space between the two locations. In some cases, the adjacent is not diagonally adjacent.

As used herein, the term “biomolecule” generally refers to any molecule that is present in living organisms or derivative thereof. Biomolecules include proteins, antibodies, peptides, enzymes, carbohydrates, lipids, nucleic acids, oligonucleotides, aptamer, primary metabolites, secondary metabolites, and natural products.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “nucleic acid sequence” or “nucleotide sequence” as used herein generally refers to nucleic acid molecules with a given sequence of nucleotides, of which it may be desired to know the presence or amount. The nucleotide sequence can comprise ribonucleic acid (RNA) or DNA, or a sequence derived from RNA or DNA. Examples of nucleotide sequences are sequences corresponding to natural or synthetic RNA or DNA including genomic DNA and messenger RNA. The length of the sequence can be any length that can be amplified into nucleic acid amplification products, or amplicons, for example, up to about 20, 50, 100, 200, 300, 400, 500, 600, 700, 800, 1,000, 1,200, 1,500, 2,000, 5,000, 10,000 or more than 10,000 nucleotides in length, or at least about 20, 50, 100, 200, 300, 400, 500, 600, 700, 800, 1,000, 1,200, 1,500, 2,000, 5,000, 10,000 or 10,000 nucleotides in length.

The term “template” as used herein generally refers to individual polynucleotide molecules from which another nucleic acid, including a complementary nucleic acid strand, can be synthesized by a nucleic acid polymerase. In addition, the template can be one or both strands of the polynucleotides that are capable of acting as templates for template-dependent nucleic acid polymerization catalyzed by the nucleic acid polymerase. Use of this term should not be taken as limiting the scope of the present disclosure to polynucleotides which are actually used as templates in a subsequent enzyme-catalyzed polymerization reaction. The template can be an RNA or DNA. The template can be cDNA corresponding to an RNA sequence. The template can be DNA.

As used herein, “amplification” of a template nucleic acid generally refers to a process of creating (e.g., in vitro) nucleic acid strands that are identical or complementary to at least a portion of a template nucleic acid sequence, or a universal or tag sequence that serves as a surrogate for the template nucleic acid sequence, all of which are only made if the template nucleic acid is present in a sample. Typically, nucleic acid amplification uses one or more nucleic acid polymerase and/or transcriptase enzymes to produce multiple copies of a template nucleic acid or fragments thereof, or of a sequence complementary to the template nucleic acid or fragments thereof. In vitro nucleic acid amplification techniques are may include transcription-associated amplification methods, such as Transcription-Mediated Amplification (TMA) or Nucleic Acid Sequence-Based Amplification (NASBA), and other methods such as Polymerase Chain Reaction (PCR), Reverse Transcriptase-PCR (RT-PCR), Replicase Mediated Amplification, and Ligase Chain Reaction (LCR).

As used herein, the term “transposome” generally refers to a complex that comprises an integration enzyme such as an integrase or transposase, and a nucleic acid comprising an integration recognition site, such as a transposase recognition site. In some examples, the transposase can form a functional complex with a transposase recognition site that is capable of catalyzing a transposition reaction. The transposase may bind to the transposase recognition site and insert the transposase recognition site into a target nucleic acid in a process sometimes termed “tagmentation.” In some examples, one strand of the transposase recognition site may be transferred into the target nucleic acid. In some examples, a transposome may comprise a dimeric transposase comprising two subunits, and two non-contiguous transposon sequences. In some examples, a transposome may comprise a dimeric transposase comprising two subunits, and a contiguous transposon sequence.

Transposases may include, but are not limited to Mu, TnlO, Tn5, hyperactive Tn5 See Goryshin and Reznikoff, J. Biol. Chem., 273:7367 (1998). Some examples can include the use of a hyperactive Tn5 transposase and a Tn5-type transposase recognition site. See Goryshin and Reznikoff, J. Biol. Chem., 273:7367 (1998). Some examples can include a MuA transposase and a Mu transposase recognition site comprising R1 and R2 end sequences. See, Mizuuchi, K., Cell, 35: 785, 1983; Savilahti, H, et al., EMBO J., 14: 4893, 1995. For example, a transposase recognition site that forms a complex with a hyperactive Tn5 transposase (e.g., EZ-Tn5™ Transposase, Epicentre Biotechnologies, Madison, Wis.) may comprise the following 19b transferred strand (mosaic end or “ME”) and non-transferred strands: 5′ AGATGTGTATAAGAGACAG 3′,5′ CTGTCT CTTATACACATCT 3′, respectively.

As used herein, the term “film” generally refers to a layer or coating having one or more constituents, applied in a generally uniform manner over the entire surface of a substrate, for example, by spin coating. In some cases, a film is a solution, suspension, dispersion, emulsion, or other acceptable form of a chosen polymer. In some cases, a film can include a photoacid generator, an acid scavenger, a sensitizer, and a matrix (a film-forming polymer). Matrices or film-forming polymers are polymers, which after melting or dissolution in a compatible solvent, can form a uniform film on a substrate.

As used herein, the term “PAG” or “photoacid generator” generally refers to any photoacid generators appropriately selected from known photoacid generators used in a conventional photo resist. Examples of the photoacid generators include, but are not limited to, onium salts, dicarboximidyl sulfonate esters, oxime sulfonate esters, diazo(sulfonyl methyl) compounds, disulfonyl methylene hydrazine compounds, nitrobenzyl sulfonate esters, biimidazole compounds, diazomethane derivatives, glyoxime derivatives, β-ketosulfone derivatives, disulfone derivatives, nitrobenzylsulfonate derivatives, sulfonic acid ester derivatives, imidoyl sulfonate derivatives, halogenated triazine compounds, equivalents thereof or combinations thereof. Onium salt photoacid generators may comprise, without limitation, alkyl sulfonate anions, substituted and unsubstituted aryl sulfonate anions, fluoroalkyl sulfonate anions, fluoarylalkyl sulfonate anions, fluorinated arylalkyl sulfonate anions, hexafluorophosphate anions, hexafluoroarsenate anions, hexafluoroantimonate anions, tetrafluoroborate anions, equivalents thereof or combinations thereof.

Some examples of photoacid generators are triphenylsulfonium trifluoromethanesulfonate, triphenylsulfonium nonafluoro-n-butanesulfonate, triphenylsulfonium perfluoro-n-octanesulfonate, and triphenylsulfonium 2-(bicyclo[2.2.1]heptan-2-yl)-1,1,2,2-tetrafluoroethanesulfonate, 4-cyclohexylphenyldiphenylsulfonium trifluoromethanesulfonate, 4-cyclohexylphenyldiphenylsulfonium nonafluoro-n-butanesulfonate, 4-cyclohexylphenyldiphenylsulfonium perfluoro-n-octanesulfonate, 4-cyclohexylphenyldiphenylsulfonium 2-(bicyclo[2.2.1]heptan-2-yl)-1,1,2,2-tetrafluoroethanesulfonate, 4-methanesulfonylphenyldiphenylsulfonium trifluoromethanesulfonate, 4-methanesulfonylphenyldiphenylsulfonium nonafluoro-n-butanesulfonate, 4-methanesulfonylphenyldiphenylsulfonium perfluoro-n-octanesulfonate, and 4-methanesulfonylphenyldiphenylsulfonium 2-(bicyclo[2.2.1]heptan-2-yl)-1,1,2,2-tetrafluoroethanesulfonate, diphenyliodonium trifluoromethanesulfonate, diphenyliodonium nonafluoro-n-butanesulfonate, diphenyliodonium perfluoro-n-octanesulfonate, diphenyliodonium 2-(bicyclo[2.2.1]heptan-2-yl)-1,1,2,2-tetrafluoroethanesulfonate, bis(4-t-butylphenyl)iodonium trifluoromethanesulfonate, bis(4-t-butylphenyl)iodonium nonafluoro-n-butanesulfonate, bis(4-t-butylphenyl)iodonium perfluoro-n-octanesulfonate, bis(4-t-butylphenyl)iodonium 2-(bicyclo[2.2.1]heptan-2-yl)-1,1,2,2-tetrafluoroethanesulfonate, 1-(4-n-butoxynaphthalen-1-yl)tetrahydrothiophenium trifluoromethanesulfonate, 1-(4-n-butoxynaphthalen-1-yl)tetrahydrothiophenium nonafluoro-n-butanesulfonate, 1-(4-n-butoxynaphthalen-1-yl)tetrahydrothiophenium perfluoro-n-octanesulfonate, 1-(4-n-butoxynaphthalen-1-yl)tetrahydrothiophenium 2-(bicyclo[2.2.1]heptan-2-yl)-1,1,2,2-tetrafluoroethanesulfonate, 1-(6-n-butoxynaphthalen-2-yl)tetrahydrothiophenium trifluoromethanesulfonate, 1-(6-n-butoxynaphthalen-2-yl)tetrahydrothiophenium nonafluoro-n-butanesulfonate, 1-(6-n-butoxynaphthalen-2-yl)tetrahydrothiophenium perfluoro-n-octanesulfonate, 1-(6-n-butoxynaphthalen-2-yl)tetrahydrothiophenium 2-(bicyclo[2.2.1]heptan-2-yl)-1,1,2,2-tetrafluoroethanesulfonate, 1-(3,5-dimethyl-4-hydroxyphenyl)tetrahydrothiophenium trifluoromethanesulfonate, 1-(3,5-dimethyl-4-hydroxyphenyl)tetrahydrothiophenium nonafluoro-n-butanesulfonate, 1-(3,5-dimethyl-4-hydroxyphenyl)tetrahydrothiophenium perfluoro-n-octanesulfonate, 1-(3,5-dimethyl-4-hydroxyphenyl)tetrahydrothiophenium 2-(bicyclo[2.2.1]heptan-2-yl)-1,1,2,2-tetrafluoroethanesulfonate N-(trifluoromethanesulfonyloxy)bicyclo[2.2.1]hept-5-ene-2,3-dicarboximide, N-(nonafluoro-n-butanesulfonyloxy)bicyclo[2.2.1]hept-5-ene-2,3-dicarboxylmide, N-(perfluoro-n-octanesulfonyloxy)bicyclo[2.2.1]hept-5-ene-2,3-dicarboxylmide, N-[2-(bicyclo[2.2.1]heptan-2-yl)-1,1,2,2-tetrafluoroethanesulfonyloxy]bicyclo[2.2.1]hept-5-ene-2,3-dicarboxylmide, N-[2-(tetracyclo[4.4.0.12,5.17,10]dodecan-3-yl)-1,1-difluoroethanesulfonyloxy]bicyclo[2.2.1]hept-5-ene-2,3-dicarboxylmide, 1,3-dioxoisoindolin-2-yl trifluoromethanesulfonate, 1,3-dioxoisoindolin-2-yl nonafluoro-n-butane sulfonate, 1,3-dioxoisoindolin-2-yl perfluoro-n-octane sulfonate, 3-dioxoisoindolin-2-yl 2-(bicyclo[2.2.1]heptan-2-yl)-1,1,2,2-tetrafluoroethanesulfonate, 3-dioxoisoindolin-2-yl N42-(tetracyclo[4.4.0.12,5.17,10]dodecan-3-yl)-1,1-difluoroethanesulfonate, 1,3-dioxo-1H-benzo[de]isoquinolin-2(3H)-yl trifluoromethanesulfonate, 1,3-dioxo-1H-benzo[de]isoquinolin-2(3H)-yl nonafluoro-n-butane sulfonate, 1,3-dioxo-1H-benzo[de]isoquinolin-2(3H)-yl perfluoro-n-octanesulfonate, 1,3-dioxo-1H-benzo[de]isoquinolin-2(3H)-yl 2-(bicyclo[2.2.1]heptan-2-yl)-1,1,2,2-tetrafluoroethanesulfonate, or 1,3-dioxo-1H-benzo[de]isoquinolin-2(3H)-yl N-[2-(tetracyclo[4.4.0.12,5.17,10]dodecan-3-yl)-1,1-difluoroethanesulfonate, (E)-2-(4-methoxystyryl)-4,6-bis(trichloromethyl)-1,3,5-triazine, 2-(Methoxyphenyl)-4,6-bis-(trichloromethyl)-s-triazine, 2-[2-(Furan-2-yl)ethenyl]-4,6-bis(trichloromethyl)-s-triazine, 2-[2-(5-methylfuran-2-yl]ethenyl)-4,6-bis(trichloromethyl)-s-triazine, 2-[2-(3,4-Dimethoxyphenyl)ethenyl]-4,6-bis(trichloromethyl)-s-triazine, equivalents thereof or combinations thereof. In some cases, photoacid generators capable of generating perfluoroalkanesulfonic acid having a high acid strength are used as the PAG in the formulations of the present disclosure. Such photoacid generators include, but are not limited to, photoacid generators capable of generating partially fluorinated alkane sulfonic acids, fully fluorinated alkane sulfonic acids, perfluorohexanesulfonic acid, perfluorooctanesulfonic acid, perfluoro-4-ethylcyclohexanesulfonic acid, perfluoroalkyl ether sulfonic acids, and perfluorobutanesulfonic acid.

As used herein, the term “photosensitizer” or “initiator synergist” generally refers to photosensitive compounds capable of absorbing light and transferring the absorbed energy to the photoacid generator. Generally speaking, a photosensitizer may expand the photosensitize wavelength band of the active energy beam of the photoacid generator. Examples of photosensitizer may include anthracene, N-alkyl carbazole, and thioxanthone compounds. Photosensitizer may include, but are not limited to, anthracenes {anthracene, 9,10-dibutoxyanthracene, 9,10-dimethoxyanthracene, 2-ethyl-9,10-dimethoxyanthracene, 2-tert-butyl-9,10-dimethoxyanthracene, 2,3-dimethyl-9,10-dimethoxyanthracene, 9-methoxy-10-methylanthracene, 9,10-diethoxyanthracene, 2-ethyl-9,10-diethoxyanthracene, 2-tert-butyl-9,10-diethoxyanthracene, 2,3-dimethyl-9,10-diethoxyanthracene, 9-ethoxy-10-methylanthracene, 9,10-dipropoxyanthracene, 2-ethyl-9,10-dipropoxyanthracene, 2-tert-butyl-9,10-dipropoxyanthracene, 2,3-dimethyl-9,10-dipropoxyanthracene, 9-isopropoxy-10-methylanthracene, 9,10-dibenzyloxyanthracene, 2-ethyl-9,10-dibenzyloxyanthracene, 2-tert-9,10-dibenzyloxyanthracene, 2,3-dimethyl-9,10-dibenzyloxyanthracene, 9-benzyloxy-10-methylanthracene, 9,10-di-α-methylbenzyloxyanthracene, 2-ethyl-9,10-di-α-methylbenzyloxyanthracene, 2-tert-9,10-di-α-methylbenzyloxyanthracene, 2,3-dimethyl-9,10-di-α-methylbenzyloxyanthracene, 9-(α-methylbenzyloxy)-10-methylanthracene, 9,10-diphenylanthracene, 9-methoxyanthracene, 9-ethoxyanthracene, 9-methylanthracene, 9-bromoanthracene, 9-methylthioanthracene, 9-ethylthioanthracene, and the like}; pyrene; 1,2-benzanthracene; perylene; tetracene; coronene; thioxanthones {thioxanthone, 2-methylthioxanthone, 2-ethylthioxanthone, 2-chlorothioxanthone, 2-isopropylthioxanthone, 2,4-diethylthioxanthone, and the like}; phenothiazine; xanthone; naphthalenes {1-naphthol, 2-naphthol, 1-methoxynaphthalene, 2-methoxynaphthalene, 1,4-dihydroxynaphthalene, 1,5-dihydroxynaphthalene, 1,6-dihydroxynaphthalene, 2,7-dihydroxynaphthalene, 2,7-dimethoxynaphthalene, 1,1′-thiobis(2-naphthol), 1,1′-bis-(2-naphthol), 4-methoxy-1-naphthol, and the like}; ketones {dimethoxyacetophenone, diethoxyacetophenone, 2-hydroxy-2-methyl-1-phenylpropan-1-one, 4′-isopropyl-2-hydroxy-2-methylpropiophenone, 2-hydroxymethyl-2-methylpropiophenone, 2,2-dimethoxy-1,2-diphenylethan-1-one, p-dimethylaminoacetophenone, p-tert-butyldichloroacetophenone, p-tert-butyltrichloroacetophenone, p-azidobenzalacetophenone, 1-hydroxycyclohexyl phenyl ketone, benzoin, benzoin methyl ether, benzoin ethyl ether, benzoin isopropyl ether, benzoin n-dibutyl ether, benzoin isobutyl ether, 1-[4-(2-hydroxyethoxy)phenyl]-2-hydroxy-2-methyl-1-propan-1-one, benzophenone, methyl o-benzoylbenzoate, Michler's ketone, 4,4′-bisdiethylaminobenzophenone, 4,4′-dichlorobenzophenone, 4-benzoyl-4′-methyldiphenylsulfide, and the like}; carbazoles {N-phenylcarbazole, N-ethylcarbazole, poly-N-vinylcarbazole, N-glycidylcarbazole, and the like}; chrysenes {1,4-dimethoxychrysene, 1,4-diethoxychrysene, 1,4-dipropoxychrysene, 1,4-dibenzyloxychrysene, 1,4-di-α-methylbenzyloxychrysene, and the like}; and phenanthrenes {9-hydroxyphenanthrene, 9-methoxyphenanthrene, 9-ethoxyphenanthrene, 9-benzyloxyphenanthrene, 9,10-dimethoxyphenanthrene, 9,10-diethoxyphenanthrene, 9,10-dipropoxyphenanthrene, 9,10-dibenzyloxyphenanthrene, 9,10-di-α-methylbenzyloxyphenanthrene, 9-hydroxy-10-methoxyphenanthrene, 9-hydroxy-10-ethoxyphenanthrene.

As used herein, the term “acid scavenger” or “amine quencher” or “amine base” generally refers to an amine base to quench the acid generated to improve the form and stability of the photoresist pattern. The acid scavenger may be a tertiary aliphatic amine or a hindered amine. Examples of the acid scavenger include, but are not limited to 2,2,6,6-tetramethyl-4-piperidyl stearate, 1,2,2,6,6-pentamethyl-4-piperidyl stearate, 2,2,6,6-tetramethyl-4-piperidyl benzoate, bis(2,2,6,6-tetramethyl-4-piperidyl) sebacate, bis(1,2,2,6,6-tetramethyl-4-piperidyl) sebacate, bis(1-octoxy-2,2,6,6-tetramethyl-4-piperidyl)sebacate, tetrakis(2,2,6,6-tetramethyl-4-piperidyl)-1,2,3,4-butanetetracarboxylate, tetrakis(1,2,2,6,6-pentamethyl-4-piperidyl)-1,2,3,4-butanetetracarboxylate, bis(2,2,6,6-tetramethyl-4-piperidyl) di(tridecyl)-1,2,3,4-butanetetracarboxylate, bis(1,2,2,6,6-pentamethyl-4-piperidyl) di(tridecyl)-1,2,3,4-butanetetracarboxylate, bis(1,2,2,4,4-pentamethyl-4-piperidyl)-2-butyl-2-(3,5-di-t-4-hydroxybenzyl)malonate, a polycondensate of 1-(2-hydroxyethyl)-2,2,6,6-tetramethyl-4-piperidinol and diethyl succinate, a polycondensate of 1,6-bis(2,2,6,6-tetramethyl-4-piperidylamino)hexane and 2,4-dichloro-6-morpholino-s-triazine, a polycondensate of 1,6-bis(2,2,6,6-tetramethyl-4-piperidylamino)hexane and 2,4-dichloro-6-t-octylamino-s-triazine, 1,5,8,12-tetrakis[2,4-bis(N-butyl-N-(2,2,6,6-tetramethyl-4-piperidyl)amino)-s-triazin-6-yl]-1,5,8,12-tetraazadodecane, 1,5,8,12-tetrakis[2,4-bis(N-butyl-N-(1,2,2,6,6-pentamethyl-4-piperidyl)amino)-s-triazin-6-yl]-1,5,8-12-tetraazadodecane, 1,6,11-tris[2,4-bis(N-butyl-N-(2,2,6,6-tetramethyl-4-piperidyl)amino)-s-triazin-6-yl]aminoundecane, and 1,6,11-tris[2,4-bis(N-butyl-N-(1,2,2,6,6-pentamethyl-4-piperidyl)amino)-s-triazin-6-yl]aminoundecane.

As used herein, the term “substantially,” when describing a relative value, a relative amount or a relative degree between two subjects, generally refers to within 90%, 91%, 92%, 93%, 94%, 95%, 96%, 97%, 98%, 99%, 100%, 101%, 102%, 103%, 104%, 105%, 106%, 107%, 108%, 109%, or 110% of each other in value, amount or degree.

As used herein, the term “matrix” or “matrices” generally refers to polymeric materials that may provide sufficient adhesion to the substrate when the photoresist formulation is applied to the top surface of the substrate, and may form a substantially uniform film when dissolved in a solvent and spread on top of a substrate. The matrices may include, but are not limited to, polyester, polyimide, polyethylene naphthalate (PEN), polyvinyl chloride (PVC), polymethylmethacrylate (PMMA) and polycarbonate, or a combination thereof. The matrix may be chosen based on the wavelength of the radiation used for the generation of acid when using the photoresist formulation, the adhesion properties of the matrix to the top surface of the substrate, the compatibility of the matrix to other components of the formulation, and the ease of removable or degradation (if needed) after use.

As used herein, the term “solvent” generally refers to an organic solution to apply the photoresist on the top surface of the substrate during coating. The solvent may help spread other components of the formulation as a substantially uniform film, for example, a thin film, on the top surface of the substrate during coating and subsequent steps. The solvent may include, but is not limited to, methyl ethyl ketone, ethyl lactate, propylene glycol methyl ether acetate (PGMEA), propylene glycol ethyl ether acetate, amyl acetate, or ethyl ether propionate (EEP), or a combination thereof.

Sequence information of nucleic acids may be the foundation to improve people's lives through clinical approaches or by material approaches. (See, Ansorge, W., “Next-generation DNA sequencing techniques,” New Biotech. (2009) 25(4):195-203, which is entirely incorporated herein by reference). Several parallel DNA sequencing platforms have been available on the market. The availability of NGS accelerates biological and biomedical research enables the comprehensive analysis of genomes, transcriptomes and interactomes. (See, Shendure, J. and Ji, H., “Next-generation DNA sequencing,” Nature Biotech. (2008) 26:1135-45, which is entirely incorporated herein by reference). One particular challenge faced by researchers in the NGS filed is a more robust protocol for generating a set of sequencing samples, for example, a set of barcoded samples.

Commonly used and commercially available NGS sequencing platforms include the Illumina Genome Analyzer, the Roche (454) Genome Sequencer, the Life Technologies SOLiD platform, and real-time sequencers such as Pacific Biosciences. Most of these platforms require the construction of a set of DNA fragments from a biological sample. The DNA fragments are, in most cases, flanked by platform-specific adapters. Common methods for constructing such a set of DNA fragments can include operations, such as, fragmenting sample DNA's, polishing ends of fragments, ligating adapter sequences to ends, selecting fragment size, amplifying fragments by PCR, and quantitating the final sample products for sequencing. The insert size or the size of the target DNA fragments in the final set of sequencing samples is a key parameter for NGS analysis.

### DNA Zipcode Array Design

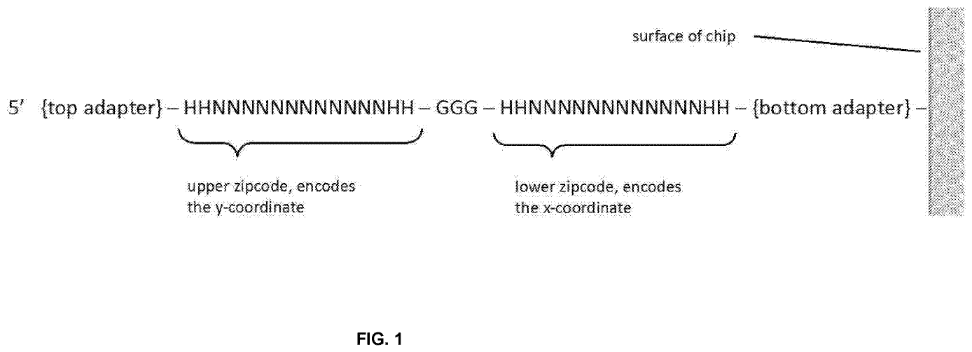

Zipcode arrays can be manufactured using a conventional contact photolithography process. In some cases, an area (about 10 mm×10 mm) of DNA zipcode array can be manufactured with features about 2 μm in size, i.e., an area of 2 μm×2 μm of identical DNA barcodes with precision alignment. In some cases, about 25 million unique zipcodes on a microarray can be made using photo-directed synthesis. Each zipcode oligo may conform to DNA barcode design requirements, such as, for example, about 40-60% GC content, no homopolymer runs, no self-complimentary stretches longer than 3 bases, and not present in human genome reference. Each zipcode oligo may comprise an upper zipcode (also called “upper barcode”) at the 5′ end and a lower zipcode (also called “lower barcode”) at the 3′ end, with the upper and lower zipcodes separated by a ‘GGG’ sequence, as illustrated in FIG. 1. In this example, the top adapter is at the 5′ end of each zipcode sequence; the bottom adapter is at the 3′ end of each zipcode sequence and is attached to the surface of a chip; a sequence of GGG separates the upper zipcode and the lower zipcode; the upper zipcode encodes the y-coordinate of the zipcode sequence; the lower zipcode encodes the x-coordinate of the zipcode sequence, the x- and y-coordinates determines the spatial location of the zipcode sequence on the zipcode array. As used herein, the term “coordinate” generally refers to numerical values or symbolic representations of a specific position on a 2-dimensional surface or in a 3-dimensional body. For example, a 2-dimensional surface can be defined according to X and Y coordinates according to a coordinate system, wherein the X and Y coordinates are the horizontal and vertical addresses of any position or addressable point, respectively. The bottom and top adapters in FIG. 1 may comprise universal sequences that are required for DNA tagging and NGS library steps that follow.

A stretched DNA may be laid on the zipcode array of a physical DNA chip. Then the stretched DNA can be cut into a plurality of fragments. Both ends of each DNA fragment may be attached to the zipcodes in the vicinity of each end, respectively. The DNA fragments and their attached zipcodes may be amplified and sequenced. After sequencing the DNA fragments the zipcode of each DNA fragment can be used to map back to an exact X-Y coordinate location on the physical DNA chip.

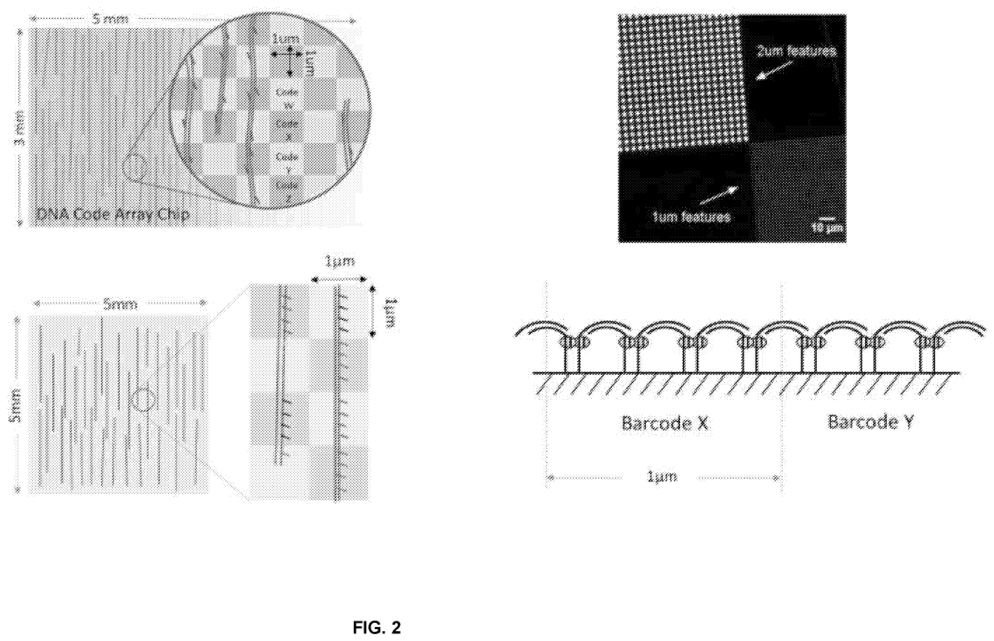

FIG. 2 shows an example of megabase sequencing. In this example, a zipcode array chip may be provided in the top left panel. Long nucleic acids may be stretched and placed on top of the zipcode array chip. The zipcode array chip (e.g., 5 mm×3 mm in size) may distinguish physical locations up to 1 μm×1 μm dimensions, i.e., all zipcodes within the 1 μm×1 μm dimension are the same, but are different from neighboring 1 μm×1 μm dimensions. The lower left panel shows another configuration of the zipcode array chip, which is 5 mm×5 mm in size, comprising 1 μm×1 μm distinctive positions encoded by zipcodes. The top right panel shows a picture of a zipcode array chip having 1 μm×1 μm distinctive positions (or features) and another zipcode array chip having 2 μm×2 μm distinctive positions (or features). The bottom panel shows an example of dissection of a zipcode array chip with barcodes X within one distinctive position (or feature) and barcodes Y within another distinctive position (or feature).

### High-Resolution Array Fabrication

De novo sequencing can be an application of the DNA zipcode arrays. In some cases, genomic DNA may be stretched and placed on top of the zipcode array. Then the zipcodes or array oligos on the zipcode array may be incorporated into the fragments of the stretched genomic DNA by various molecular biology techniques, thereby resulting in array-genomic material that can be analyzed by commercial DNA sequencers. The zipcodes or array oligos can be synthesized oligos on the microarray. As described above, the zipcodes can be designed to identify positional information for each of the fragments of the genomic DNA, thereby providing positional information for DNA fragments so that adjacent DNA fragments may be mapped. After sequencing the DNA fragments and decoding the zipcodes, the sequenced pieces can be unequivocally assembled based on the zipcode information for each fragment. In some cases, the synthesized oligos on the microarray can be resolved at 1 μm feature sizes or less, giving about 2000 bp resolution as to the location of the sequenced DNA fragments. The photoresist described herein may be formulated for providing sub-micron patterning resolution with spatial distribution of chemistry applied to each feature and for being compatible with DNA chemistries at each feature. The photoresist described herein may provide the high resolution patterns resolved at 1 μm feature sizes or less without substantial sequencing error for oligos within each feature. For example, the sequencing error for oligos within each feature may be no more than 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01%. Sequencing error for oligos may include insertions and deletions (indels) in DNA barcodes.

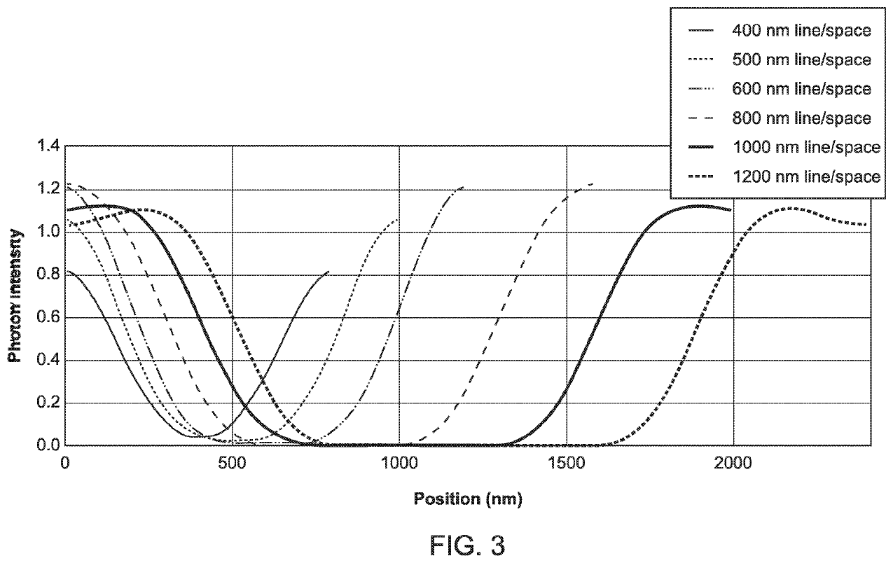

Photoresist may be formulated to synthesize DNA oligo zipcode microarray at high resolution. One factor affecting the resolution of features in an oligo zipcode microarray made by the photolithography process may be the aerial image (photon distribution at the wafer plane) emanating from common commercial steppers. FIG. 3 shows a calculated aerial image from an ASML PA/60 i-line stepper. In FIG. 3, various periodic lines and spaces (L/S) patterns from 400 nm through 1.2 μm may be modelled. X-axis is nm and Y-axis is light intensity. For the 400 nm L/S patterns, the intensity in the nominally exposed region may not reach full intensity and the light may not be completely excluded from anywhere in the nominally unexposed region. For larger features, such as, for example 700 nm L/S patterns, the light intensity may reach zero between the exposed lines. However, the gradual slope of the aerial image may be a concern if the photoresist are to produce chemistry linearly with photon intensity. For example, extra bases may be printed on a portion of the oligos outside the exposed regions in patterning because light can be available in the unexposed region due to the gradual slope of light intensity.

Accordingly, in one embodiment of the invention, photoresist formulations with high “contrast” may be chosen. As used herein, the term “high contrast” when describing a photoresist formulation generally refers to formulations that print no chemistry or substantially little chemistry when receiving less than the full amount of light for chemistry, but rapidly switch to full chemistry upon receiving the full amount of light for chemistry. As used herein, the term “full chemistry” generally refers to a chemical reaction or chemical reactions that are triggered by sufficient light exposure or chemicals generated by sufficient light exposure.

TABLE 1
Composition of “low amine, low ITX” formulation

| Components | Weight (g) | % wt/wt |
|---|---|---|
| PAG | 0.53 | 2.6 |
| Photosensitizer | 0.28 | 1.3 |
| Acid scavenger | 0.024 | 0.12 |
| Matrix | 0.72 | 3.5 |
| Solvent | 19.2 | 92.5 |

TABLE 2
Composition of “high amine, low ITX” formulation

| Components | Weight (g) | % wt/wt |
|---|---|---|
| PAG | 0.71 | 2.7 |
| Photosensitizer | 0.38 | 1.5 |
| Acid scavenger | 0.06 | 0.23 |
| Matrix | 0.94 | 3.6 |
| Solvent | 24.1 | 92.0 |

TABLE 3
Composition of “low amine, high ITX” formulation

| Components | Weight (g) | % wt/wt |
|---|---|---|
| PAG | 0.92 | 3.9 |
| Photosensitizer | 0.93 | 3.9 |
| Acid scavenger | 0.037 | 0.16 |
| Matrix | 0.82 | 3.5 |
| Solvent | 21 | 88.6 |

TABLE 4
Composition of “high amine, high ITX” formulation

| Components | Weight (g) | % wt/wt |
|---|---|---|
| PAG | 0.56 | 4.1 |
| Photosensitizer | 0.56 | 4.1 |
| Acid scavenger | 0.048 | 0.35 |
| Matrix | 0.48 | 3.5 |
| Solvent | 12 | 87.9 |

TABLE 5
Composition of “mid amine, mid ITX” formulation

| Components | Weight (g) | % wt/wt |
|---|---|---|
| PAG | 16.1 | 3.4 |
| Photosensitizer | 15.35 | 3.2 |
| Acid scavenger | 0.96 | 0.20 |
| Matrix | 15.27 | 3.2 |
| Solvent | 427.7 | 90.0 |

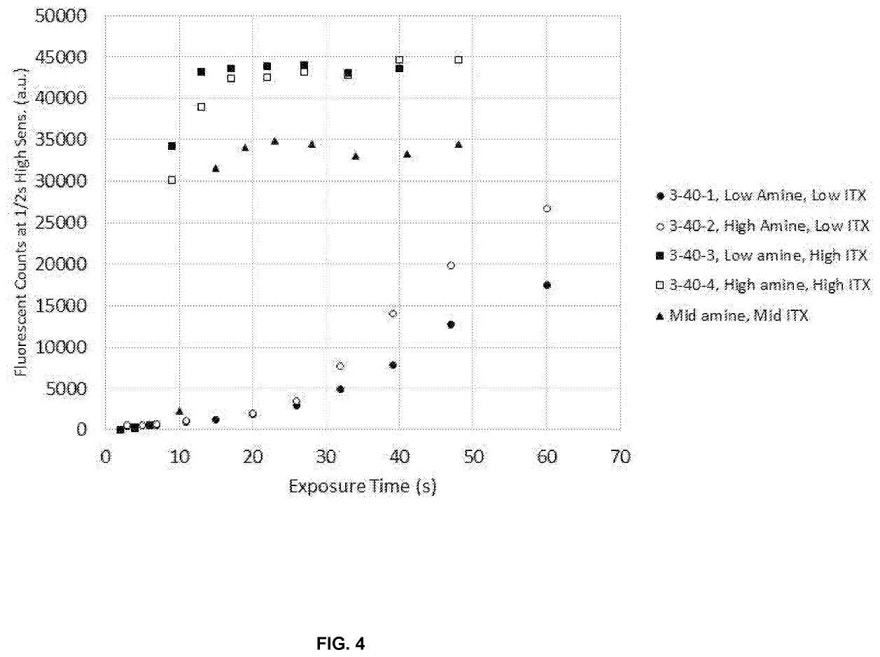

Examples of low contrast vs. high contrast formulations is shown in FIG. 4, where a 2×2 design of experiments (DOE) may be performed for acid scavenger (also called “amine quencher”) and photosensitizer (also called “initiator synergist” or ITX). For the compositions listed in Tables 1-5, the following components may be used: the photoacid generator (PAG) may be bis(4-tert-butylphenyl)iodonium perfluoro-1-butanesulfonate (BBI-PFBS); the photosensitizer may be 2-isopropylthioxanthone (ITX); the acid scavenger may be 1,2,2,6,6-Pentamethyl-4-piperidinol; the matrix may be poly(methyl methacrylate) (PMMA, molecular weight about 35 k); and the solvent may be propylene glycol monomethyl ether acetate (PGMEA).

“Contrast” curve measurements may be recorded after varying any components, for example, varying two variables of a photoresist formulation. In these examples in FIG. 4, two components, i.e., the concentrations of the acid scavenger (also called amine quencher) and the photosensitizer (e.g., ITX) may be changed. In this case, the formulations with high ITX can show high “contrast”. At low doses of light (measured by the exposure time in second, no more than 9 seconds), all formulations may show reasonable response (i.e., the doses of light may not print chemistry, shown in FIG. 4). However, formulations with high ITX concentrations may show a greater “contrast” than the ones with low ITX concentrations when the full dose of light for print chemistry has been delivered (shown in FIG. 4 at or near 20 second of exposure time). In this case, high “contrast” formulations may lead to very high print chemistry within a small change in photon intensity close to the required full dose (or mount) of light, and hence may lead to finer features printed from the aerial images shown in FIG. 3. Further, when the photoresist absorbs small amounts of light (i.e., below or substantially below the threshold of the required dose (amount) of light for chemistry), the photoresist may not produce a chemical response. Hence, the corresponding high “contrast” formulations may print, for example, the 400 nm L/S patterns shown in FIG. 3, with no chemistry between the printed lines, and may sharpen the transition between the nominally exposed and nominally unexposed regions.

To obtain different “contrast” level for the photoresist formulations, many factors can be changed or optimized. These factors include, for example, photoacid generators, photosensitizers (initiator synergists), acid scavengers (amine quenchers), matrices (substrate), and solvents. High “contrast” formulations (i.e., those behave similarly to the high ITX formulations in FIG. 4) can be used to print chemistry using actual L/S patterns.

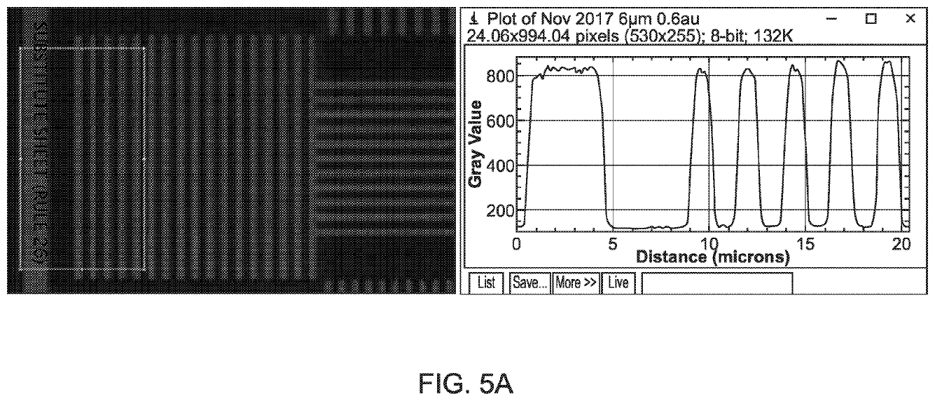

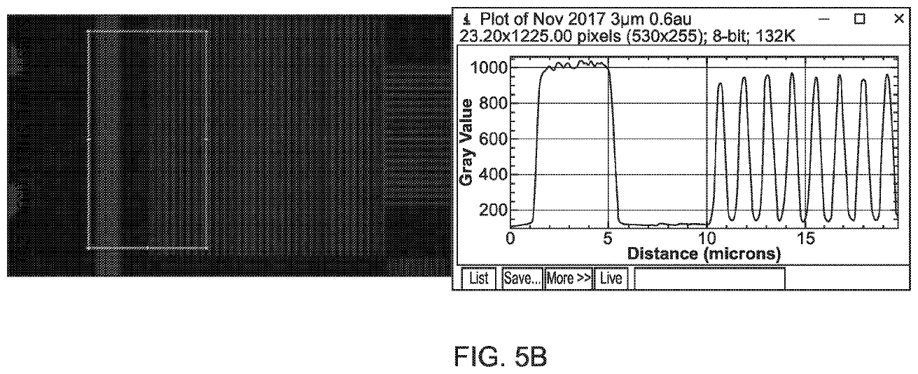

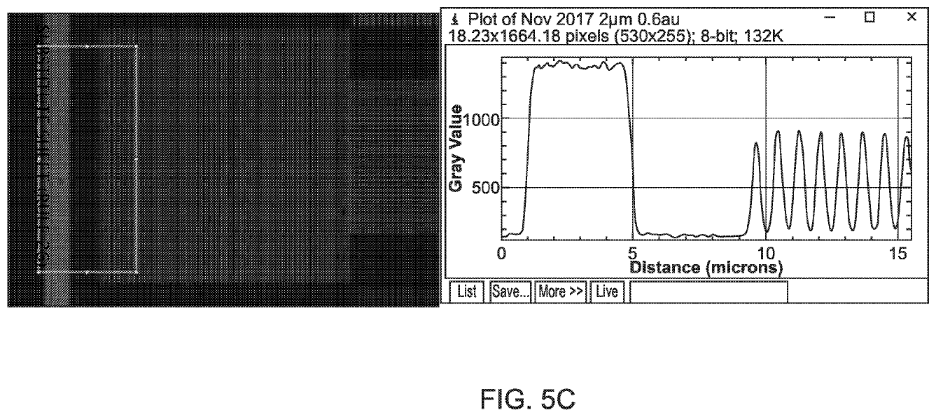

When the features can be patterned at about 1 μm resolution level using the high “contrast” formulation of the present disclosure and the patterns can be imaged using a fluorescence microscope at 100×, metrology can be used to determine the details of the images obtained. FIGS. 5A-5C show the results of pattering the same formulation of the present disclosure on the ASML PA/60 using 1.2 μm, 0.6 μm, and 0.4 m L/S patterns, respectively, all printed on the same wafer. In FIGS. 5A-5C show a single base extension experiment. In this experiment, four consecutive thymine nucleotides (4T's) may be immobilized (i.e., covalently bonded) to the surface of a substrate (e.g., a wafer) in 3′ to 5′ orientation with the terminal T at the 5′-end (i.e., the top T distal to the surface of the substrate) protected by a 4,4′-dimethoxytrityl (DMT) group on its 5′-hydroxyl. Then the photoresist may be spun on to the surface bonded with 4 T's, and the resulting substrate with photoresist may be exposed to light using a mask to generate acid in a pattern and deblock the DMT group of the top T. The freed 5′-hydroxyl groups on the top T can react with a fluorescein phosphoramidite under solid-phase oligonucleotide synthesis conditions. The substrate (i.e., the wafer with fluorescein phosphoramidite bonded to its surface) can be imaged under a fluorescence microscope.

FIG. 5A shows that the 1.2 m L/S patterns may demonstrate that the peak chemistry concentration (here represented by peak fluorescence) in the lines (shown on the right hand side) is similar to that in the bulk pattern (shown on the left hand side). FIG. 5B shows that the 600 nm L/S patterns may demonstrate that the peak chemistry concentration in the lines (shown on the right hand side) is similar to that in the bulk pattern (shown on the left hand side) without substantial differences. However, FIG. 5C shows that the 400 nm L/S patterns may demonstrate that the peak chemistry concentration in the lines (shown on the right hand side) is different from that in the bulk pattern (shown on the left hand side). The different peak chemistry concentrations for lines vs. bulk in the 400 nm L/S patterns may be due to the aerial image intensity degradation from the stepper, and may also be caused by the metrology tool.

FIG. 5C also shows the gradual slope of the transition from full chemistry to no chemistry within the L/S patterns (i.e., low “contrast”). The gradual slope of the transition may lead to some chemistry completed about 300-400 nm from where the desired chemistry should be (in other words, some chemistry happen at the wrong location). The wrong chemistry caused by the gradual slope of the transition may lead to an “insertion” base being added in features (unintended location) adjacent to the feature of interest (intended location) in a zipcode array. At the same time close to the edge of the line within the nominally exposed region, non-complete deblocking may occur due to insufficient exposure.

This lack of observed “top hat” chemical distribution (i.e., substantially constant chemistry distributions with sharp edges or steep slope) may be caused by the metrology tool. In this case, a Canon FV3000 point scanning confocal tool using 100× objective, 1.49 NA, and 0.6 Airy units on the pinhole may be cable of resolving the two adjacent features about 150-200 nm apart using the Rayleigh criterion. The images shown in FIGS. 5A-5C are without deconvolution routines. However, even with deconvolution routines an about 150 nm point spread function (psf) of the metrology tool may be convolved with the actual chemical distribution. For small features in zipcode arrays where adjacent features may be close to each other, for example, within 400 nm of each other, this uncertainty of whether chemistry is spatially defined correctly or whether the metrology tool is the problem may be problematic.

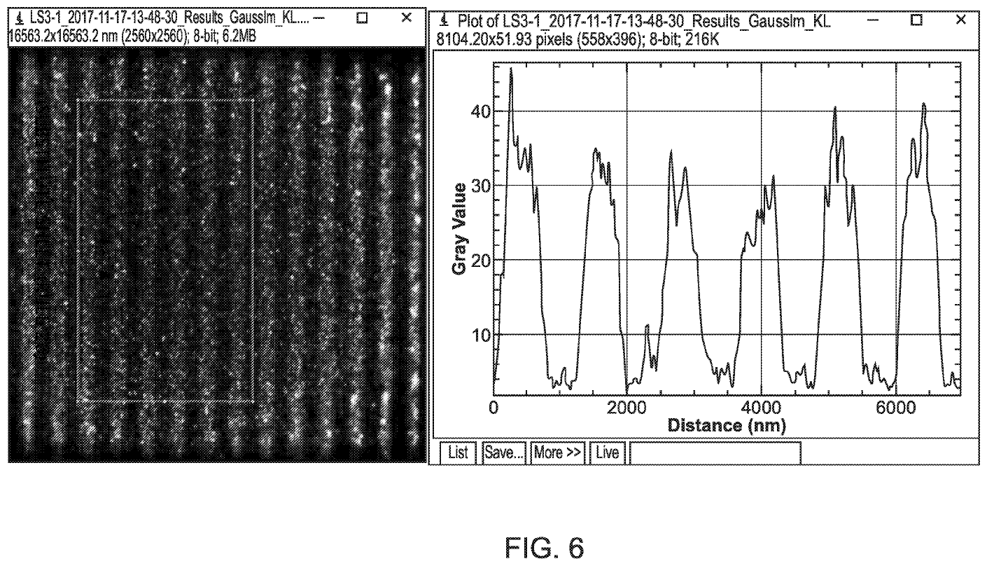

To address this uncertainty, super resolution microscopy may be used. A super resolution microscopy may assign a location to a single molecule by excluding adjacent fluorophores, and measure the psf during many blinking cycles. In this way certainty can be assigned to the center of the psf, often on the order of several tens of nm. In this case, stochastic optical reconstruction microscopy (STORM) can be employed. FIG. 6 shows results from a STORM imaging process for 600 nm L/S patterns. The image in FIG. 6 may have some artifacts from the STORM imaging and may be ameliorated with a multi-emitter routine to produce a more linear response between intensity in the image and chemistry on the wafer and remove the photo bleached region near the center of the image. Nevertheless FIG. 6 of the STORM image shows that the transition of the image from the bright areas to the dark areas may be about 100 nm in this case, indicating that the true chemical distribution may be a top hat distribution, or a trapezoid-shape distribution with about 100 nm runout on the walls instead of the 3-400 nm indicated in the point confocal images shown in FIG. 5. For a 600 nm line, identifying the true chemistry as more of a top hat distribution shape with about 100 nm runout into the nominally unexposed regions may mean that some chemistry may be present about ⅙th or about 16% into the adjacent features, while no or substantially no chemistry is absent in the normally exposed region.

Therefore, comparing the STORM image of 600 nm L/S of FIG. 6 with the confocal image of 600 nm L/S of FIG. 5B may indicate that different metrology tools may provide different images of and conclusion about the chemistry on features of no more than 1 μm in size. For example, FIG. 5B may indicate that extra (or errant) chemistry may be present nearly across the entire unexposed region, for example, may be present at about 80% of the unexposed region. FIG. 5B may also indicate that chemistry is lacking in the exposed region across about 100% of the line. Accordingly, a knowledge of the actual chemical distribution on the wafer may provide better direction for oligo printing at these fine features of no more than 1 μm in size and using super resolution microscopy of all types may be the metrology tools for characterization of such fine features.

### Formulations

In some embodiments, the photoresist formulation can be applied to the patterned oligos and a base, such as, for example, an amine base, may be added without undue damage to the oligos underneath the photoresist layer. In this case, techniques such as LC/MS, and various fluorescence/hybridization experiments can be used to show the extent of damage or the lack of damage to the oligo sequences underneath the photoresist layer.

To form the photoresist layer, formulations in poly(methyl methacrylate) (PMMA) may result in a better contrast for the oligo microarray than other matrix polymers such as polystyrene, poly(α-methylstyrene), and the like. PMMA can also be obtained in pure form without impurities, such as, for example, residual initiators, heavy metals, and the like, that may cause compatibility issues with the oligos under the photoresist.

For photoinitiators, in some cases, a very low pKa acid when released under radiation may yield high resolutions. In addition, the very low pKa acid may have low or substantially no damage to synthesized oligos with the exception of the base G. However, other acids with different pKa ranges may be useful.

For solvents, in some cases, propylene glycol methyl ether acetate (PGMEA or 1-methoxy-2-propanol acetate) and ethyl lactate may be used with consideration for safety and compatibility within standard cleanrooms such that the photoresist can be used with other formulations in a semiconductor fabrication plant (a fab; or foundry) so that existing infrastructure for manufacture can be utilized.

When choosing synergists, speed of energy transfer may be considered. In addition the compatibility of the synergist to create acid efficiently without cross-reactions from the excited state to another matrix or another DNA molecule.

Base additives may be considered based on their pKb, for their non-nucleophilicity, and for enhancing the contrast performance when combined with other components of the photoresist formulation.

### Dose Response of the Formulation

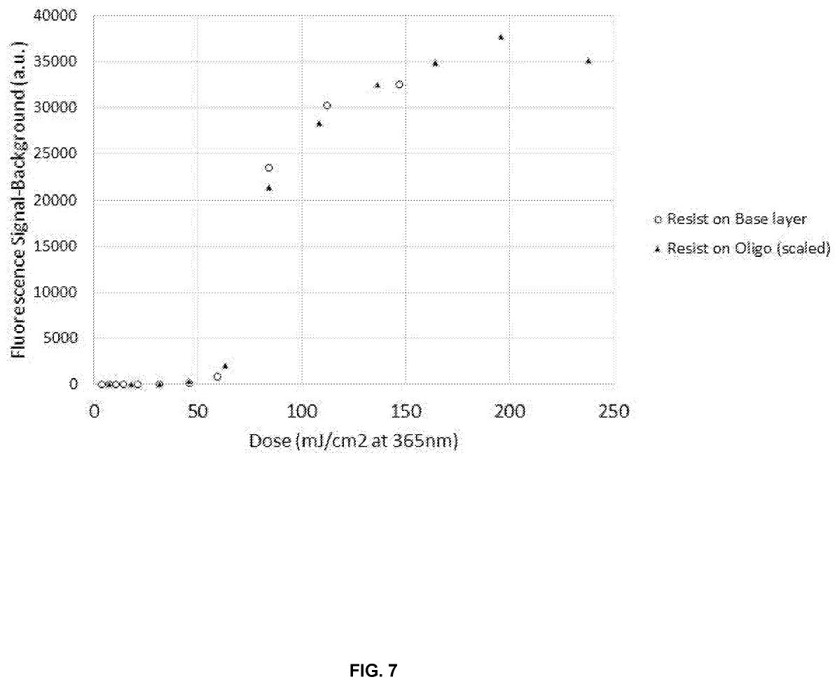

In some cases, oligonucleotide synthesis may be performed on quartz substrates using commercial DMT oligonucleotide monomers and a photo-acid generator (PAG) system. In this case, the feature size and pitch of zipcode macroarray may be reduced to sub 1 μm scale. The formulation is PGMEA-based polymer film of photoresist with optimized photo-acid generator chemistry to provide contrast enhancement in generating zipcode features. FIG. 6 shows measured dose response curves, where a print ultraviolet (UV) dose of 150 mJ/cm² may be considered to fully “print” a feature by generating acid in the photoresist film. With this print UV dose, the photo-generated acid may deprotect the underlying DMT group on the oligo from the hydroxyl group. The freed hydroxyl group may react with a labeled phosphoramidite which provides the fluorescence signal as an indication of the acid generation process. When different doses of the UV light are provided, the ensuing measurements of fluorescence may provide data points for a dose-response curve as shown in FIG. 7. As shown in FIG. 7, while a UV dose of 150 mJ/cm² (print dose) may generate substantially high concentration (or amount) of acid, a UV dose of 50 mJ/cm² may not generate detectable concentration (amount) of acid. Accordingly, if the dark areas around the feature may receive less than the required print dose for full chemistry, for example, no more than one-third of the required print dose, the dark area may remain inert (with no acid generation), thereby preventing an erroneous base from being added onto the zipcode in the dark area around the feature. The sigmoidal dose-response curves in FIG. 7 may demonstrate a sharp (or high) contrast in acid generation (gauged by the fluorescence signal observed) between the feature which may receive a print dose and the dark areas which may receive less than the print dose (e.g., one-third of the print dose).

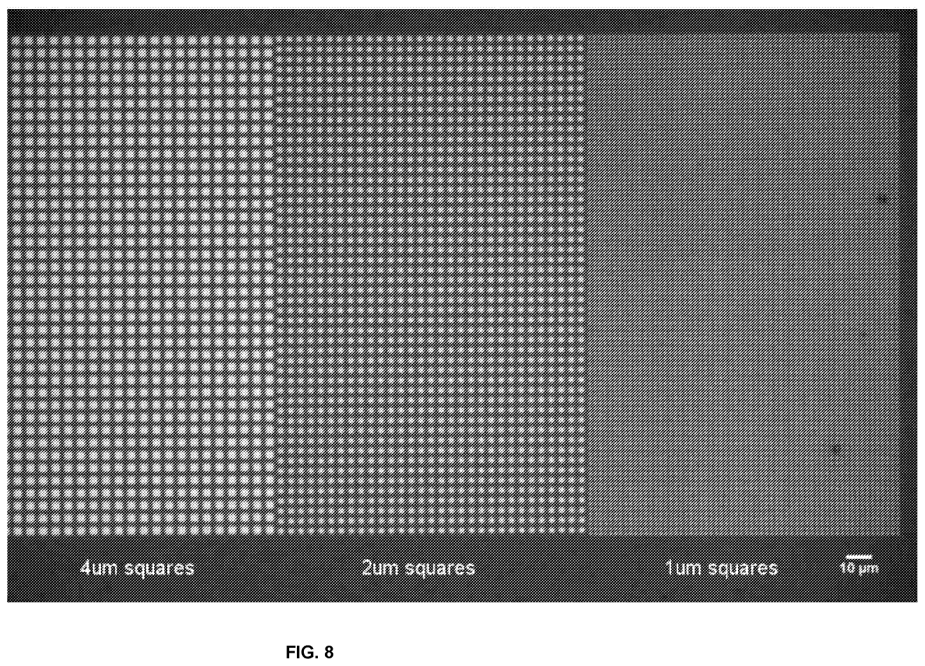

Contact printing of such contrast enhancing PAG films may produce feature arrays with resolution down to 1 μm in size (FIG. 8). FIG. 8 shows contact lithography dot resolution patterns of squares with 4 μm, 2 μm and 1 μm resolution levels on the same substrate. The 40× fluorescence image in FIG. 8 may show fluorescein isothiocyanate (FITC) labeled oligonucleotide features down to 1 μm in size.

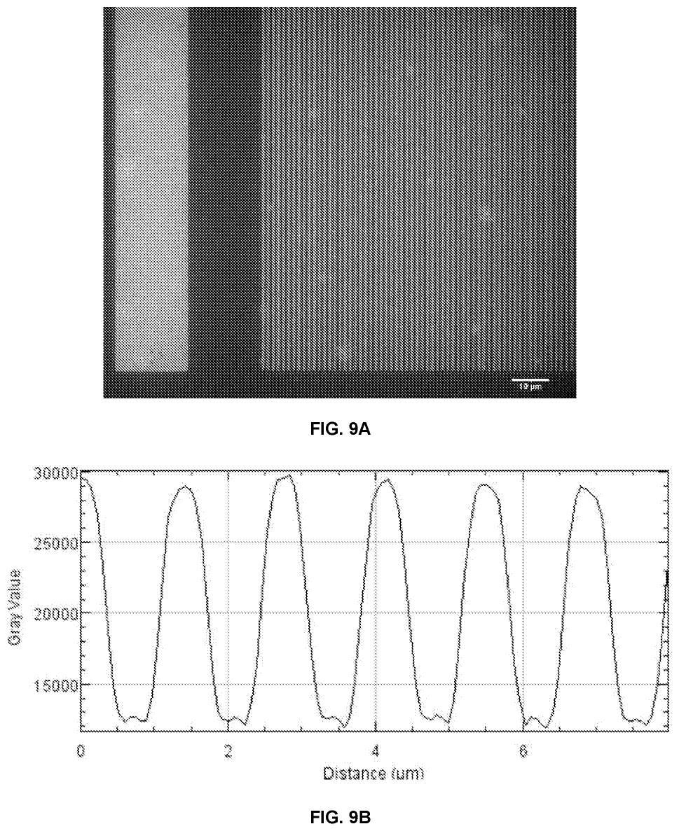

Furthermore, a projection lithography system (e.g., ASML PAS5500 at Stanford Nanofabrication Facility) may be used to project 5× reduced aerial images of feature arrays onto the spin-coated PAG film of the present disclosure. In some cases, 700 nm oligonucleotide features can be printed as demonstrated by the one-dimensional lines and spaces (L/S) pattern in FIGS. 9A-9B. FIG. 9A shows a fluorescence image (100× oil immersion) of 700 nm lines and spaces pattern of FITC labeled oligonucleotides printed via the ASML PAS5500 projection lithography system. FIG. 9B shows a cross-section of the L/S pattern cutting through the paralleled lines, indicating that the width of the features may be about 700 nm.

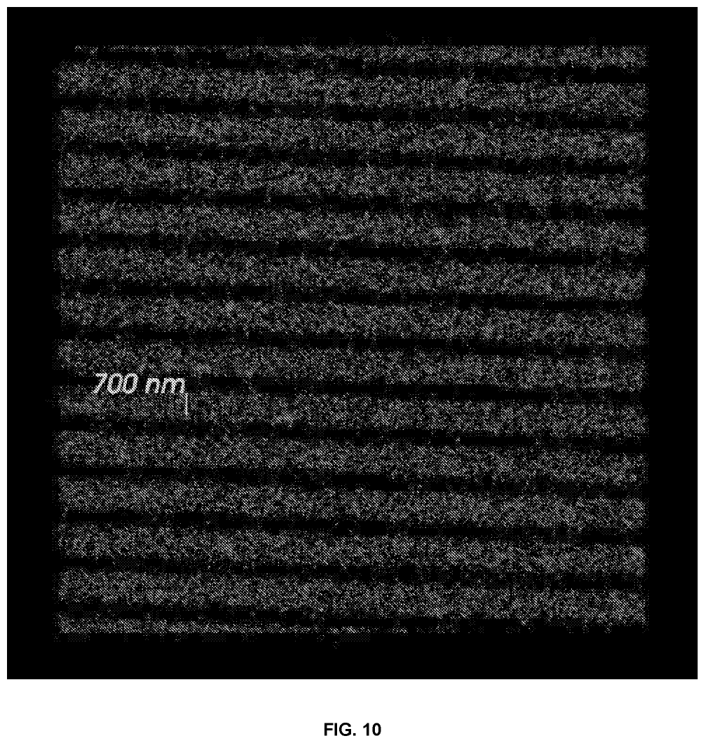

At these small scales down to about 1 μm level and below, acquisition of high-resolution images may be blurred by the diffraction limit of conventional microscope objectives. To measure the dimensions of printed patterns, the substrates may be scanned on Vutara Super Resolution Microscope (Bruker) with 60× immersion objective. Using the STORM technique, individual molecules may be imaged in FIG. 10 (dot over the background) and the composite image may depict the printed lines and spaces pattern. Analysis of the image molecular histogram may indicate a full width at half maximum (FWHM) line width of about 723 nm for FIG. 10. In this case, the oligonucleotides are labeled with Cy5 for detection.

The photoresist formulation of the present disclosure may not only provide sub-μm resolution for features on the microarray, but it may also provide chemical compatibility with the polymer-PAG chemistry and sufficient reaction yields for the ensuing printing of nucleotide(s) to the growing oligonucleotide chain. For example, the damage to the oligos due to UV and photoacid generation may be reduced no more than 1.5% per layer. Factoring in the deblock efficiency of the polymer-PAG system, the overall yield of the oligo synthesis achieved may be about 90% per layer.

In some cases, photo-cleavable groups (PCG) may be put on the 5′-OH group of phosphoramidite reagents. For example, compounds of Formula I may be used in oligonucleotide synthesis methods disclosed in the present disclosure:

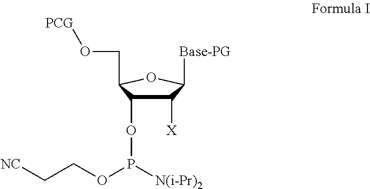

wherein PCG is a photo-cleavable group; X is H (for DNA synthesis) or a protected 2′-hydroxy group (for RNA synthesis); Base is a nucleic acid base or nucleobase including but not limited to: adenine (A), cytosine (C), guanine (G), thymine (T), and uracil (U), or analogs thereof, and PG is none, or a protecting group on reactive groups (for example, N atom or O atom) on the Base. In particular, PG may include but not be limited to N-benzoyl (Bz), N-acetyl (Ac), N-isobutyryl (iBu), N-phenoxyacetyl (PAC) and N-tert-butylphenoxyacetyl (tBPAC). Further, PCG may include but not be limited to 5′-(α-methyl-2-nitropiperonyl)oxycarbonyl (MeNPOC), 2-(2-nitrophenyl)propoxycarbonyl (NPPOC), dimethoxybenzoincarbonate (DMBOC), and thiophenyl-2-(2-nitrophenyl)-propoxycarbonyl (SPh-NPPOC), the structures of which are shown below:

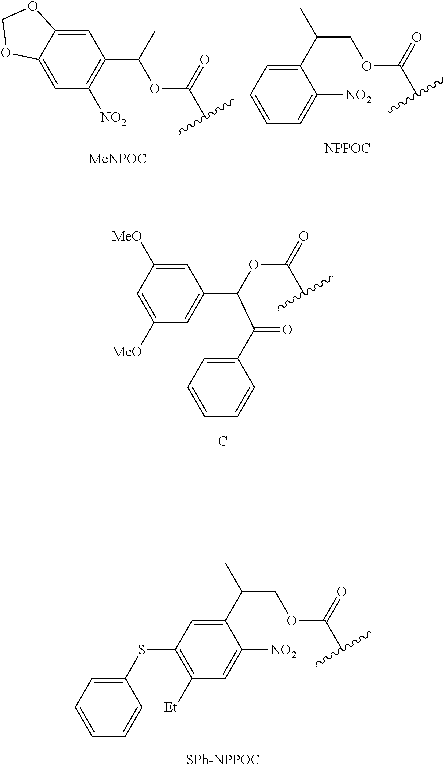

In some cases, implementation of the zipcode microarray may be a blended approach of using compounds of Formula I (up to about 97% layer yield) for majority low-resolution construction of features (larger sizes), then complemented with up to six, seven, eight, nine, or ten high-resolution polymer-PAG synthesized layers defining the smallest features.

### Example

1) Photoresist Preparation

Components for PAG “V 4.0” Photoresist:
a) Photoacid generator (PAG)
   Bis(4-tert-butylphenyl)iodonium perfluoro-1-butanesulfonate (BBI-PFBS, electronic grade, from Sigma-Aldrich):
   About 3.4% w/w, final concentration about 50 mM
b) Acid scavenger (amine quencher)
   1,2,2,6,6-Pentamethyl-4-piperidinol (Sigma-Aldrich):
   About 0.2% w/w, final concentration about 12 mM
c) Matrix (polymer)
   Poly(methyl methacrylate) (PMMA, MW about 35,000, Sigma-Aldrich):
   About 3.2% w/w
d) Photosensitizer (initiator synergist)
   2-Isopropylthioxanthone (ITX):
   About 3.2% w/w, final concentration about 125 mM
e) Solvent
   Propylene glycol monomethyl ether acetate (PGMEA)
   About 90% (balance)

Preparation Procedure:

The PMMA is dissolved in PGMEA first, as prolonged heating and stirring is required (between about 45-55° C., for about 18-36 hrs). The other components (PAG, acid scavenger and photosensitizer) are then added to the polymer solution, and dissolved by stirring at room temperature overnight to afford the PAG “V 4.0” photoresist formulation. The solution is stored at about 4° C., and used within 8 weeks of the preparation.

2) Substrate Processing:
1. Bring hot plate to temperature (about 50° C.) by turning on about 20 min. prior to the processing experiments.
2. Place photoresist mixture (e.g., the PAG “V 4.0” photoresist formulation) into a 5 mL syringe, fitted with filter and needle.
3. Place 6″ wafer on chuck of spin coater (Cee Brewer Science 200CB Photoresist Spin Coater Hot Plate Combo Tool)
4. Run program with a dispense cycle for 10 seconds at 0 rpm, a spread cycle for 10 seconds at 500 rpm (1000 acceleration), and a “thickness” cycle for 60 seconds at 1500 rpm (1000 acceleration).
   Dispense about 1.5 ml of photoresist in the 5 mL syringe onto center of wafer during the dispense cycle.
   Allow the spin coater to complete its entire cycle.
5. Remove wafer from spinner chuck. Manually remove edge bead with wipe moistened with PGMEA. If using POLOS spin coater (SPS-Europe) for single wafer spincoating, the wiping step can be done on the spin tool.
6. Place wafer on hot plate pins. Run program: 10 seconds with pins at 20 mm, 2 seconds to pins at 0 mm, and 178 seconds vacuum bake at 50° C. with lid open.
7. Remove wafer from hot plate. It is now ready for exposure on a mask aligner (Neutronix/Quintel NXQ 9000 aligner).
8. Load, align and expose wafer according to manufacturer-recommended procedure for vacuum contact mode and 36 mJ/cm² exposure dose (at 365 nm).
9. After last exposure, the substrate is allowed to rest for 4 minutes, and then rinsed with PGMEA, acetone, and isopropyl alcohol (IPA) on the spin coater, in that order, three times consecutively.
10. Wafer is transferred to synthesizer flowcell for the addition of the desired nucleoside, linker, or fluorescent phosphoramidite monomers. Standard oligonucleotide synthesis chemistry is employed as described elsewhere (G H McGall and J A Fidanza, Methods in Molecular Biology DNA Arrays Methods and Protocols, edited by J. B. Rampal Humana, Totowa, N.J., 2001, pp. 71-101.)

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for forming a pattern of oligonucleotides on a microarray, comprising:
(a) forming a photoresist layer by applying a photoresist composition onto an underlying layer of a substrate, wherein the photoresist composition comprises a photoacid generator and a photosensitizer, wherein the underlying layer comprises a plurality of functional groups protected by protective groups;
(b) exposing a dose of light through a patterned mask onto the substrate; and
(c) removing protective groups on a section of the plurality of functional groups within at least one exposed region of the substrate;
thereby forming a pattern on the substrate, wherein the pattern comprises the at least one exposed region, and wherein the at least one exposed region is no more than 1 micrometer in at least one dimension.

2. The method of claim 1, wherein the functional groups are amino or hydroxyl groups.

3. The method of claim 1, further comprising:
(d) contacting the functional groups within the at least one exposed region of the substrate with a first nucleotide reagent,
thereby coupling a fraction of the functional groups within the at least one exposed region of the substrate with a first nucleotide.

4. The method of claim 3, further comprising:
(e) exposing another dose of light through another patterned mask onto the substrate;
(f) removing protective groups on another section of the plurality of functional groups within at least another exposed region of the substrate;
thereby forming another pattern on the substrate, wherein the another pattern comprises the at least another exposed region, and wherein the at least another exposed region is no more than 1 micrometer in at least one dimension.

5. The method of claim 4, further comprising:
(g) contacting the functional groups within the at least another exposed region of the substrate with a second nucleotide reagent,
thereby coupling another fraction of the functional groups within the at least another exposed region of the substrate with a second nucleotide.

6. The method of claim 5, the first nucleotide is different from the second nucleotide.

7. The method of claim 4, the at least one exposed region is different from the at least another exposed region.

8. The method of claim 3, further comprising:
(e) forming another photoresist layer by applying another photoresist composition onto the substrate, wherein the another photoresist composition comprises another photoacid generator and another photosensitizer, wherein the underlying layer comprises a plurality of functional groups protected by protective groups;
(f) exposing another dose of light through another patterned mask onto the substrate;
(g) removing protective groups on another section of the plurality of functional groups and/or a nucleotide protective group on a nucleotide functional group on the first nucleotide within at least another exposed region of the substrate;
thereby forming another pattern on the substrate, wherein the another pattern comprises the at least another exposed region.

9. The method of claim 8, wherein the at least another exposed region is no more than 1 micrometer in at least one dimension.

10. The method of claim 8, further comprising:
(h) contacting the functional groups and/or the nucleotide functional group on the first nucleotide within the at least another exposed region of the substrate with a second nucleotide reagent,
thereby coupling another fraction of the functional groups and/or the nucleotide functional group within the at least another exposed region of the substrate with a second nucleotide.

11. The method of claim 10, the first nucleotide is different from the second nucleotide.

12. The method of claim 9, the at least one exposed region is different from the at least another exposed region.

13. The method of any one of claims 1-12, wherein the at least one exposed region is no more than 950 nm, 900 nm, 850 nm, 800 nm, 750 nm, or 700 nm in the at least one dimension.

14. The method of any one of claims 1-12, wherein weight percentage of the photosensitizer is substantially the same as weight percentage of the photoacid generator.

15. The method of claim 14, wherein the weight percentage of the photosensitizer is the same as the weight percentage of the photoacid generator.

16. The method of any one of claims 1-12, wherein the photoresist composition further comprises, an acid scavenger, a matrix and a solvent.

17. The method of any one of claims 1-12, wherein the photoresist composition comprises:
the photoacid generator: about 2-5% by weight;
the photosensitizer: about 2-5% by weight;
an acid scavenger: about 0.1-0.5% by weight;
a matrix: about 2.5-4.5% by weight; and
a solvent: about 85-93.4% by weight.

18. The method of claim 17, wherein the photoresist composition comprises:
the photoacid generator: about 2.5-4.5% by weight;
the photosensitizer: about 2.5-4.5% by weight;
the acid scavenger: about 0.15-0.35% by weight;
the matrix: about 3.0-4.0% by weight; and
the solvent: about 86.7-91.8% by weight.

19. The method of claims 17 or 18, wherein weight percentage of the photosensitizer is substantially the same as weight percentage of the photoacid generator.

20. The method of claim 19, wherein the weight percentage of the photosensitizer is the same as the weight percentage of the photoacid generator.

21. The method of any one of claims 3-20, wherein the pattern and/or the another pattern comprises features of oligonucleotides; and wherein the smallest size of the features of oligonucleotides is no more than 1 μm in at least one dimension.

22. The method of claim 21, wherein smallest size of the features of oligonucleotides is no more than 950 nm, 900 nm, 850 nm, 800 nm, 750 nm, or 700 nm in the at least one dimension.

23. The method of claim 21, wherein the features of oligonucleotides is no more than 950 nm, 900 nm, 850 nm, 800 nm, 750 nm, or 700 nm in two dimensions.

24. The method of any one of claims 1-23, wherein sizes of features of the pattern, features of the another pattern, the at least one exposed region, the at least another exposed region, and/or the features of oligonucleotides are measured by using a super resolution microscopy.

25. A method for forming a pattern of oligonucleotides on a microarray, comprising:
(a) activating a photoacid generator in the presence of a photosensitizer in selected regions, thereby producing an acid from the photoacid generator, wherein the substrate comprises a functional group protected by a protective group, wherein the protective group is removed by the acid;
(b) contacting the substrate with a reagent for oligonucleotide synthesis; and
(c) repeating steps (a) and (b) with another reagent for oligonucleotide synthesis;
thereby forming a pattern of oligonucleotides, wherein at least one feature of the pattern of oligonucleotides is no more than 1 μM in at least one dimension.

26. The method of claim 25, further comprising heating the substrate.

27. The method of claim 25, further comprising directing a light to the selected regions in step (a).

28. The method of claim 27, wherein a print dose of the light is directed to the selected region.

29. The method of claim 28, wherein the print dose of the light produce the acid from the photoacid generator.

30. The method of claim 28, wherein when no more than one-third of the print dose is directed to the selected region, another photoacid generator within the selected region does not produce another acid from the another photoacid generator.

31. The method of claim 25, further comprising an acid scavenger in step (a).

32. The method of claim 25, further comprising, prior to step (a), coating the substrate with a photoresist formulation comprising the photoacid generator and the photosensitizer.

33. The method of claim 32, wherein the photoresist formulation further comprises a matrix and a solvent.

34. The method of claim 25, wherein the at least one feature of the pattern of oligonucleotides comprises a plurality of features of oligonucleotides.

35. The method of any one of claims 25-34, wherein the selected region, and/or the plurality of features of oligonucleotides are no more than 1 μM in at least one dimension.

36. The method of claim 35, wherein the selected region, the at least one feature of the pattern of oligonucleotides, and/or the plurality of features of oligonucleotides are no more than 950 nm, 900 nm, 850 nm, 800 nm, 750 nm, or 700 nm in the at least one dimension.

37. The method of claim 36, wherein the selected region, the at least one feature of the pattern of oligonucleotides, and/or the plurality of features of oligonucleotides are no more than 950 nm, 900 nm, 850 nm, 800 nm, 750 nm, or 700 nm in two dimensions.

38. The method of any one of claims 32, 33, and 35-37, wherein step (a) is conducted using a spin coater.

39. The method of any one of claims 25-38, wherein step (b) is conducted by using an oligonucleotide synthesizer.

40. The method of any one of claims 25-39, wherein weight percentage of the photosensitizer is substantially the same as weight percentage of the photoacid generator.

41. The method of claim 40, wherein the weight percentage of the photosensitizer is the same as the weight percentage of the photoacid generator.

42. A photoresist composition comprises:
a photoacid generator: about 2-5% by weight;
a photosensitizer: about 2-5% by weight;
an acid scavenger: about 0.1-0.5% by weight;
a matrix: about 2.5-4.5% by weight; and
a solvent: about 85-93.4% by weight.

43. The photoresist composition of claim 42, wherein:
the photoacid generator: about 2.5-4.5% by weight;
the photosensitizer: about 2.5-4.5% by weight;
the acid scavenger: about 0.15-0.35% by weight;
the matrix: about 3.0-4.0% by weight; and
the solvent: about 86.7-91.8% by weight.

44. The photoresist composition of claim 42 or claim 43, wherein weight percentage of the photosensitizer is substantially the same as weight percentage of the photoacid generator.

45. The photoresist composition of claim 42 or claim 43, wherein the weight percentage of the photosensitizer is the same as the weight percentage of the photoacid generator.
